(19) United States
(12) Plant Patent Application Publication
(10) Pub. No.: US 2003/0115644 P1
(43) Pub. Date: Jun. 19, 2003

(54) Fiona's delight Hybrid tea rose

(76) Inventor: Ronald Rosenberg, Holland, MI (US)

Correspondence Address: Michael Berns, Maloney Parkinson & Berns, 135 West Main Street, Urbana, IL, 61801, US

(21) Appl. No.: 10/016,012

(22) Filed: Dec. 17, 2001

(51) Int. Cl. A01H 5/00

(52) U.S. Cl. PLT/137

(57) ABSTRACT

A new variety of hybrid tea rose plant moderate in size and with sturdy stems, having a seven-day blooming cycle where its color changes from a coral-pink to white and its form change from a split-centered to a rosette shape.

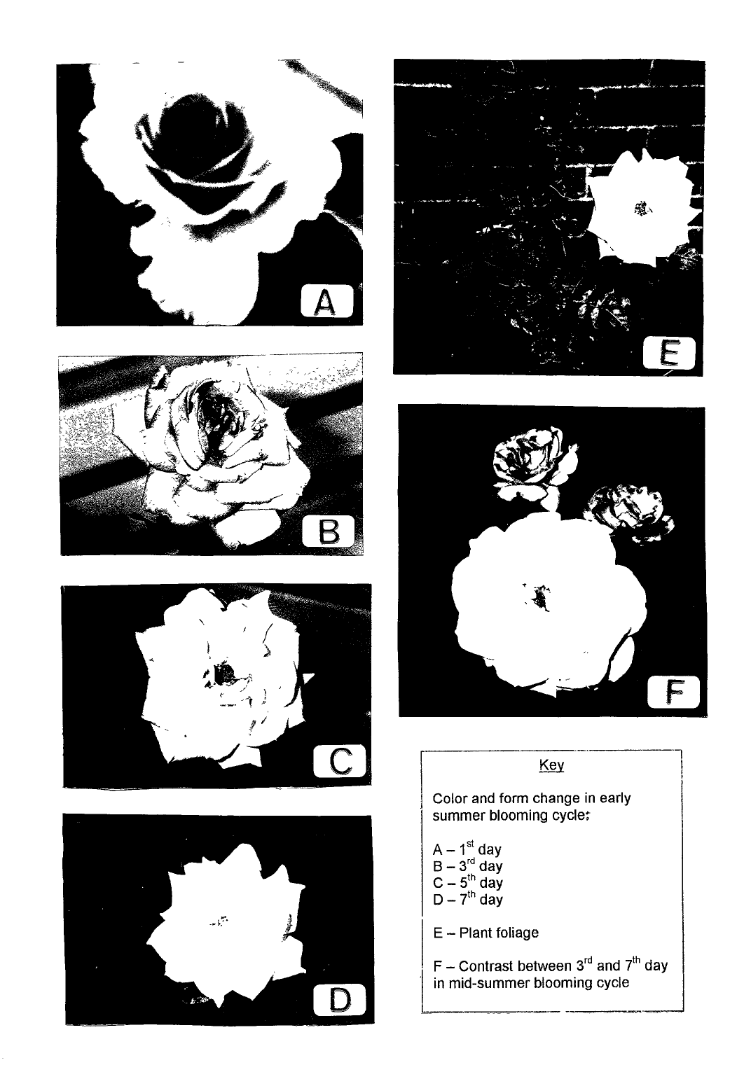

The present invention relates to a new hybrid tea rose plant, moderate in size and with sturdy stems, bearing slightly semi-glossy green foliage. Winter protection in some areas is advisable and mildew in late summer is possible. Its obvious distinction is that in a seven-day blooming cycle, its color changes from a coral-pink to white, and its form changes from a split-centered to a rosette shape.

The resulting plant has been designated “Fiona's Delight” and registered with the American Rose Society. The objective of this breeding was to cross white roses with colored roses to develop a white rose with a colored edge.

It is a coral-pink split-centered rose that changes to a rosette-shaped white rose with a pink edge. This was achieved by crossing Honor (Jacolite, Michele Torr, U.S. Pat. No. PP 4167) with Cherish (Jascal, U.S. Pat. No. PP4331).

The following unique combination of characteristics are outstanding in the new variety and distinguish it from its parents, as well as from all other varieties of which I am aware:

1. Novel color change from coral-pink to white;

2. Form change from split-centered to rosette shape;

3. Sturdy stems;

4. Moderate fragrance;

5. Moderate size, and;

6. Semi-glossy green foliage.

Over a three year study, the garden plants were carefully monitored in the moderate temperature range of Western Michigan, near Holland, Mich. The positive qualities inherited from good parent stock, together with its unique color and form change, make it a very attractive rose.

Asexual reproduction of this new variety by budding, as performed at Holland, Mich., shows that the foregoing and all other characteristics and distinctions come true to form and are established and transmitted through succeeding propagation.

The attached drawing illustrates the new plant as herein describe. Figure A, B, C, and D show day-to-day color and form change of the early summer blooming cycle. Figure E shows the plant foliage. Figure F shows the contract between the third and seventh day on the mid-summer blooming cycle.

Following is a detailed description of the new rose sing color descriptions as defined by The Royal Horticultural Society (London) Color Chart and using proper botanical terms or commonly accepted plant terminology when practical.

Classification:

Botanical.—Rosa Hybrida.

Commercial.—Hybrid Tea.

Parentage:

As described by “Taylor's Guide to Roses”

Honor.—(‘Jacolite’, ‘Michele Torr’), by Warriner (1980) Seed Parent U.S. Pat. No. PP4167

White, double petals.—20-22, all-season bloom, slight fragrance, high centered form, upright, vigorous, well-branched, disease resistant, needs winter protection, large dark green leaves.

Cherish.—(‘Jascal’) by Warriner (1980) Pollen Parent U.S. Pat. No. PP 4331.

Coral-pink, double petals.—28, good bloom, regular cycles, spicy fragrance, blooms centered, spreading vigorous bushes, disease resistant, some winter protection needed, leaves are dark green and semi-glossy.

Both Honor and Cherish won 1980 All-American Rose Awards.

Plant:

General appearance and growth development.—Own root bush, height — 80 cm, width — 50 cm, moderately full slightly semi-glossy foliage. Center stems a bit high in proportion, but a very sturdy bush with erect stems. Consistent three cycles per season of blooming. Color and form change also very consistent in three years of growth. Tendency to mildew in late summer. Some winter protection advisable. Cultivar would benefit in plant width by being budded on rootstock.

Wood:

Generally slightly prickled, main stem, old wood.—Green Group 137B. Main stem, new wood — Purple Group N77D with shades of Green Group 138A.

Thorns:

Shape.—Varied, some straight, some slightly concave at the end.

Quantity.—Eighteen on 10 cm of main stem, irregular sizes range from 5 mm to 8 mm.

Color.—New growth — Greyed Red Group 178A. Old growth — Greyed Orange Group 166A.

Foliage:

Leaflets.—Number of leaves usually five, 16 cm long, 11 cm wide, leaves oval in shape. Terminal leaf 6 cm long, 4.5 cm wide. Serration, small. Texture smooth and slightly glossy, stipules — bearded, 2 cm in length. Older leaflets somewhat larger.

Color.—New foliage — Yellow Green Group 147A on upper surface, Purple Group N77! with Green Group 138B on lower surface. Old foliage — upper surface Green Group 139A, lower surface Green Group 138A. Petiole-rhachis — Old growth, Yellow Green Group 147B. New growth, Purple Group 77A.

Reproduction:

Pollen.—Yellow Orange Group 18A.

Filaments.—Not tightly grouped, 1.5 cm long. Color — Red Purple Group N57C shading to Orange Group 27B.

Anthers.—Yellow Orange Group 16B.

Styles and stigma.—Green Yellow Group 1D.

Inflorescence (bud):

Size.—3 to 4 cm wide at base, 4 to 5 cm long, form is triangular and slightly curved.

Color.—At first color show, inside petals — Red Purple Group 58C, outside petals — Red Purple Group 62C. At third day of color show, inside petals — Red Purple Group 58B, outside petals remain Red Purple Group 62C.

Sepals.—Size 6 cm long.

Color.—Green Group 138B on outside surface, Green Group 138C on inside surface, some sepals shade to White Group N155D on attached end.

Receptacle.—Urn shaped — 2 cm wide, 1.5 cm long.

Color.—Green Group 138B.

Peduncle.—A bit long in proportion, most stems 10 cm long.

Surface.—slightly rough.

Color.—Yellow Green Group 14C shaped to Grey-Eyed Group 182C.

Florescence (full flowering):

Color and form described together.—

First Day

Form of corolla is split-centered with a tightly curled center opening completely on the outer edges to a slightly curled outer edge. Basic color is commonly called coral-pink. Petal numbers range from 20 to 30. Flower width ranges from 10.5 cm to 11 cm. Inner petals — Red Purple Group 68A, both sides the same color. Average length 2.5 cm. Outside petals — Red Purple Group 62B shading to Red Purple Group 65D, both sides the same color. 5 cm long.

Third Day

Form beginning to change toward a rosette shape. About 12 inner petals separating. Inner petals — inner edge, Red Purple Group 62A, outer edge Red Purple Group 62D. Outer petals — Red Purple Group 62D with Red Purple Group 62B edges. Colors the same on both sides.

Fifth Day

Form continues to more or a rosette shape with less curled edges. Inner petals — Red Purple Group 69A shading to Red Purple Group 69D. Outer petals — Red Purple Group 69D with Red Purple Group 69A edge. Color the same on both sides.

1. A new variety of hybrid tea rose plant described herein with slightly semi-glossy green foliage on sturdy stems, and characterized by both its color change from a coral-pink to white and its form change from a split-centered to a rosette shape in a seven-day blooming cycle.
